(19) United States
(12) Patent Application Publication
(10) Pub. No.: US 2024/0293152 A1
(43) Pub. Date: Sep. 5, 2024

(54) COAXIAL GUIDE NEEDLE FOR PUNCTURE TO PREVENT GAS EMBOLISM

(71) Applicant: Mailin Chen, Beijing (CN)

(72) Inventor: Mailin Chen, Beijing (CN)

(21) Appl. No.: 18/332,615

(22) Filed: Jun. 9, 2023

(30) Foreign Application Priority Data
Mar. 3, 2023 (CN) 2023101979846

(51) Int. Cl. A61B 17/34

(52) U.S. Cl. CPC A61B 17/3498; A61B 17/3421; A61B 2017/3454; A61B 2017/3456

(57) ABSTRACT

A coaxial guide needle for puncture to prevent gas embolism includes a needle sheath handle, a needle sheath, a needle core handle, an anti-gas valve and a needle core, an end of the needle sheath handle is in sealed connection with the needle sheath, another end of the needle sheath handle is used for connecting the needle core handle, the needle core handle is connected with an end of the needle core, another end of the needle core is disposed through the needle sheath; the needle sheath handle is provided with the anti-gas valve therein, and an edge of the anti-gas valve is in sealed connection on an inner wall of the needle sheath handle. A problem that air exposure of percutaneous coaxial guide needle causes gas embolism is solved, a safety of percutaneous technology is significantly improved, the percutaneous technology is promoted and economic and social benefits are improved.

### TECHNICAL FIELD

The disclosure relates to the field of puncture coaxial guides, and more particularly to a coaxial guide needle for puncture to prevent gas embolism.

### BACKGROUND

Percutaneous coaxial guide needles have been an important auxiliary tool of clinical operations such as percutaneous guided puncture biopsy, drainage, radiofrequency ablation, and seed implantation technique in clinical treatment. The percutaneous coaxial guide needle is guided to a target area through a needle core puncture, and a convenient channel is established between the target area in a body and the external body through a coaxial outer sheath, corresponding operations can be repeated through the channel, which is more conducive to diagnosis and treatment. However, due to establishment of the channel and a pressure difference effect between atmospheric pressure and internal pressure, a probability of air entering the body greatly increases, if the air enters damaged blood vessels, a gas embolism will be caused, which will lead to poor prognoses and even death.

An application of the percutaneous coaxial guide needle is increasing with a widely development of various percutaneous techniques in the clinical treatment, reports of being complicated by the gas embolism and causing death are gradually increasing in recent years. In addition to technical and cognitive factors of operators, a main reason of them is a design defect of the percutaneous coaxial guide needle at present. During operations of percutaneous guided puncture biopsy, ablation, and seed implantation, the channel established by the coaxial outer sheath has a risk of air exposure, and the air can easily enter the body, so as to cause the gas embolism.

### SUMMARY

A purpose of the disclosure is to provide a coaxial guide needle for puncture to prevent gas embolism, so as to solve problems mentioned in the above background.

In order to achieve the above purpose, the disclosure provides the following technique solutions.

A coaxial guide needle for puncture to prevent gas embolism, includes a needle sheath handle, a needle sheath, a needle core handle, an anti-gas valve and a needle core, an end of the needle sheath handle is in sealed connection with a tail end of the needle sheath, another end of the needle sheath handle is configured to connect the needle core handle, the needle core handle is connected with a tail end of the needle core, a head end of the needle core is disposed through the needle sheath; an inner of the needle sheath handle is provided with the anti-gas valve, an edge of the anti-gas valve is in sealed connection on an inner wall of the needle sheath handle.

In an embodiment, the head end of the handle core is a punctured triangular tip.

In an embodiment, the needle core handle is threadedly connected to another end of the needle sheath handle.

In an embodiment, a coaxial position of the anti-gas valve with the needle sheath is provided with a cross-shaped gap, a length of each edge of the cross-shaped gap is smaller than a radius of the needle core; and the cross-shaped gap is configured to guide the needle core when the needle core is inserted and fit a surface of the needle core after expanding of the anti-gas valve.

In an embodiment, the needle sheath and the needle sheath handle are hollow tubular structures, an outer surface of the needle sheath is provided with a graduated scale, a head end of the needle sheath is a blunt tip.

Compared with related art, beneficial effects of the disclosure are as follows.

The purpose of the disclosure is to solve a problem that air exposure of percutaneous coaxial guide needle may cause gas embolism, a safety of percutaneous technology is significantly improved. The disclosure is conducive to promoting and popularizing the percutaneous technology, improving economic benefits and social benefits.

### BRIEF DESCRIPTION OF DRAWINGS

FIG. 1 illustrates a schematic structural diagram of a coaxial guide needle for puncture to prevent gas embolism according to an embodiment of the disclosure.

FIG. 2 illustrates a schematic sectional structural diagram of a coaxial guide needle for puncture to prevent gas embolism according to an embodiment of the disclosure.

FIG. 3 illustrates a schematic structural diagram of a needle sheath handle and a needle sheath of a coaxial guide needle for puncture to prevent gas embolism according to an embodiment of the disclosure.

FIG. 4 illustrates a schematic structural diagram of a needle core handle and a needle core of a coaxial guide needle for puncture to prevent gas embolism according to an embodiment of the disclosure.

FIG. 5 illustrates a schematic structural diagram of an anti-gas valve of a coaxial guide needle for puncture to prevent gas embolism according to an embodiment of the disclosure.

FIG. 6 illustrates a schematic structural diagram of a needle core through an anti-gas valve of a coaxial guide needle for puncture to prevent gas embolism according to an embodiment of the disclosure.

### DETAILED DESCRIPTION OF EMBODIMENTS

The technical solutions in embodiments of the disclosure will be clearly and completely described in conjunction with the accompanying drawings. The described embodiments are only some of the embodiments of the disclosure, not all of them. Based on the embodiments in the disclosure, all other embodiments acquired by those skilled in the art without creative work fall within the scope of protection of the disclosure.

In order to make the above purposes, features and advantages in the disclosure more obvious and understandable, the following will provide a further detailed explanation of the disclosure in conjunction with the drawings and the embodiments.

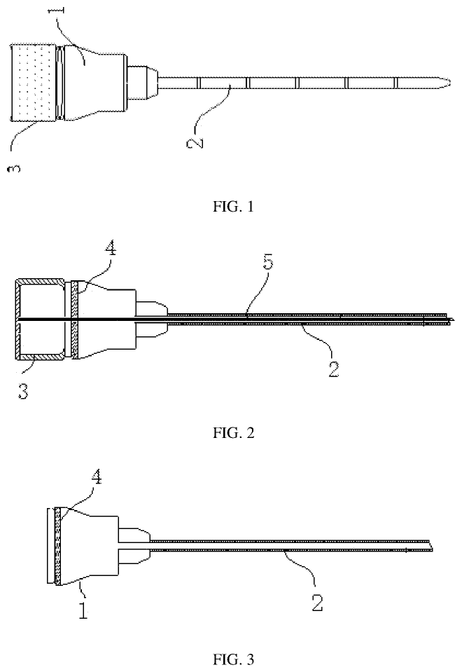

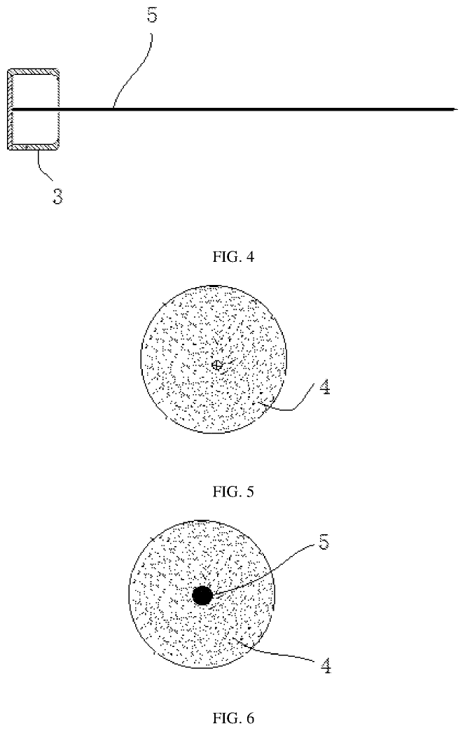

Please refer to FIGS. 1-7, in the embodiment of the disclosure, a coaxial guide needle for puncture to prevent gas embolism includes a needle sheath handle 1, a needle sheath 2, a needle core handle 3, an anti-gas valve 4 and a needle core 5,

An end of the needle sheath handle 1 is in sealed connection with a tail end of the needle sheath 3, another end of the needle sheath handle 1 is configured to connect the needle core handle 3, the needle core handle 3 is connected with a tail end of the needle core 5, a head end of the needle core 5 is disposed through the needle sheath 2. The head end of the handle core 5 is a punctured triangular tip or a blunt tip.

The needle core handle 3 is threadedly connected on an opening of another end of the needle sheath handle 1.

An inner of the needle sheath handle 1 is provided with the anti-gas valve 4, and an edge of the anti-gas valve 4 is in sealed connection on an inner wall of the needle sheath handle 1. A coaxial position of the anti-gas valve 4 with the needle sheath 2 is provided with a cross-shaped gap (as shown in FIG. 5), a length and a width of the cross-shaped gap are slightly smaller than a diameter of puncture needle (i.e., needle core 5). Specifically, the cross-shaped gap is cut by a blade, in a natural state of the cut cross-shaped gap, characteristics of rubber material ensure the cross-shaped gap remain sealed. A length of each edge of the cross-shaped gap is smaller than a radius of the needle core 5, thus the cross-shaped gap can guide when the needle core 5 is inserted and can better fit a surface of the needle core 5 after expanding, meanwhile, a thickness of the anti-gas valve 4 is limited, and the thickness of the anti-gas valve 4 need to ensure that during a puncture progress, the puncture needle pushes and squeezes the anti-gas valve 4, a displacement of the anti-gas valve 4 in a horizontal direction can be ignored, the cross-shaped gap of the anti-gas valve 4 can slide tightly against an outer wall of the puncture needle during the puncture progress, thus ensuring air tightness. Therefore, a size ratio of the anti-gas valve needs to be selected according to actual needs, a conventional selection is the diameter to the thickness ratio of 5:1 for the anti-gas valve 4. The cross-shaped gap of the anti-gas valve 4 can be provided with a hard rubber ring, the hard rubber ring is tightly attached to the inner wall of the needle sheath handle 1 to provide a certain support force for the anti-gas valve 4, so as to avoid significant deformation of the anti-gas valve 4 due to a pushing effect of the puncture needle, resulting air intake in the cross-shaped gap.

The needle sheath 2 and the needle sheath handle 1 are hollow tubular structures, an outer surface of the needle sheath 2 is provided with a graduated scale, a head end of the needle sheath 2 is a blunt tip.

A structure provided in the disclosure is to add the anti-gas valve based on traditional coaxial guide needle, the anti-gas valve is made of a natural rubber material with good elasticity, meanwhile the rubber needs to be performed desulfurization treatment to ensure the rubber is non-toxic, and the anti-gas valve is integrated with the needle sheath handle. A center of the anti-gas valve 4 has a pinhole (such as cross-shaped gap), when the needle core is not inserted, the anti-gas valve is closed, when the needle core is inserted, the anti-gas valve is tightly attached to the needle core, the anti-gas valve has good needle core compatibility and smoothness, making it easier to insert and exit the needle core. In practical operation, whether inserting or pulling out the needle core, a needle sheath channel is always in a closed and isolated air state, so that air cannot enter body through the needle sheath channel, avoiding a possibility of the gas embolism.

Meanwhile, in order to balance service life, consumables, and economic effects of the guide needle, the anti-gas valve 4 can be made into a movable and detachable form to avoid aging of the anti-gas valve 4 after multiple uses, which affects the air tightness of the device.

Specifically, the inner wall of the needle sheath handle 1 is provided with an annular baffle, the annular baffle is provided with an annular groove, a side of the anti-gas valve 4 is provided with an annular insert block, and an interference fit is adopted between the anti-gas valve 4 and the needle sheath handle 1. A material of the annular insert block is made of a hard rubber block, a frictional strength between the annular insert block and the annular baffle is ensured, and the interference fit is adopted between the annular insert block and the annular groove, thus improving a fit strength between the anti-gas valve 4 and the annular insert block and ensuring the air tightness of the device. A connection between the annular insert block and the annular baffle is achieved by squeezing, a strengthen of the anti-gas valve 4 is improved by effect of the annular insert block at the same time, and a strain of the anti-gas valve 4 is reduced. After aging of the anti-gas valve 4, the anti-gas valve can be taken out through a device such as a needle-nose plier, adhesive residue in the annular baffle, drug solution in the gap and the like are cleared, the anti-gas valve 4 is replaced to a new one, so that consumables can be greatly reduced and economic benefits of the device can be improved. The device has a wide range of use, and the anti-gas valve 4 can be applied to all puncture needles, such as puncture needles for radio frequency, freezing, microwave, particle implantation, and the like.

In the description of the disclosure, it is necessary to understand that the terms “direction”, “transverse”, “up”, “down”, “front”, “back”, “left”, “right”, “vertical”, “horizontal”, “top”, “bottom”, “inside”, “outside”, and the like indicate orientation or positional relationships based on the orientation or positional relationships shown in the accompanying drawings and are intended only to facilitate the description of the invention, not to indicate or imply that the device or element referred to must have a particular orientation or be constructed and operate in a particular orientation, and therefore are not to be construed as limiting the disclosure.

The above-described embodiments only describe the preferred way of the disclosure, do not limit the scope of the disclosure. Without departing from the spirit of the design of the disclosure, all kinds of deformations and improvements made to the technical solutions of the disclosure by those skilled in the art shall fall within the scope of protection determined by the claims of the disclosure.

What is claimed is:

1. A coaxial guide needle for puncture to prevent gas embolism, comprising a needle sheath handle, a needle sheath, a needle core handle, an anti-gas valve and a needle core, wherein an end of the needle sheath handle is in sealed connection with a tail end of the needle sheath, another end of the needle sheath handle is configured to connect the needle core handle, the needle core handle is connected with a tail end of the needle core, a head end of the needle core is disposed through the needle sheath; an inner of the needle sheath handle is provided with the anti-gas valve, an edge of the anti-gas valve is in sealed connection on an inner wall of the needle sheath handle.

2. The coaxial guide needle for puncture to prevent gas embolism as claimed in claim 1. wherein the head end of the handle core is a punctured triangular tip.

3. The coaxial guide needle for puncture to prevent gas embolism as claimed in claim 1, wherein the needle core handle is threadedly connected to another end of the needle sheath handle.

4. The coaxial guide needle for puncture to prevent gas embolism as claimed in claim 1, wherein a coaxial position of the anti-gas valve with the needle sheath is provided with a cross-shaped gap, a length of each edge of the cross-shaped gap is smaller than a radius of the needle core; and the cross-shaped gap is configured to guide the needle core when the needle core is inserted and fit a surface of the needle core after expanding of the anti-gas valve.

5. The coaxial guide needle for puncture to prevent gas embolism as claimed in claim 1, wherein the needle sheath and the needle sheath handle are hollow tubular structures, an outer surface of the needle sheath is provided with a graduated scale, a head end of the needle sheath is a blunt tip.

6. The coaxial guide needle for puncture to prevent gas embolism as claimed in claim 4, wherein the anti-gas valve is made of a rubber material.

7. The coaxial guide needle for puncture to prevent gas embolism as claimed in claim 4. wherein a ratio of a diameter to a thickness of the anti-gas valve is 5:1.

8. The coaxial guide needle for puncture to prevent gas embolism as claimed in claim 4, wherein the anti-gas valve is provided with a rubber ring, the rubber ring is tightly attached to the inner wall of the needle sheath handle to provide a support force for the anti-gas valve.

9. The coaxial guide needle for puncture to prevent gas embolism as claimed in claim 4, wherein the anti-gas valve is detachably connected to the needle sheath handle.

10. The coaxial guide needle for puncture to prevent gas embolism as claimed in claim 9, wherein the inner wall of the needle sheath handle is provided with an annular baffle, the annular baffle is provided with an annular groove, the anti-gas valve is provided with an annular insert block, and the annular insert block is in interference fit with the annular groove.

11. The coaxial guide needle for puncture to prevent gas embolism as claimed in claim 1, wherein the tail end of the needle core is configured to penetrate through the needle sheath, the needle sheath handle and the anti-gas valve to connect with the needle core handle, and the anti-gas valve is tightly attached to the needle core.
